(19) United States
(12) Patent Application Publication
(10) Pub. No.: US 2010/0104630 A1
(43) Pub. Date: Apr. 29, 2010

(54) WATER SOLUBLE STATIN MICROSTRUCTURES AND METHOD OF PREPARATION

(76) Inventors: Donald E. Ackley, Cardiff, CA (US); Eugene Tu, San Diego, CA (US)

Correspondence Address: ROBERT A. PARSONS, 4000 N. CENTRAL AVENUE, SUITE 1220, PHOENIX, AZ, 85012, US

(21) Appl. No.: 12/607,403

(22) Filed: Oct. 28, 2009

Related U.S. Application Data
(60) Provisional application No. 61/109,013, filed on Oct. 28, 2008.

(51) Int. Cl. A61K 9/127; A61K 31/40; A61P 9/00

(52) U.S. Cl. 424/450; 514/423

(57) ABSTRACT

A method of preparing a water soluble microstructure includes the steps of providing a saturated solution of a payload with poor water solubility, providing a solution of helper molecules, and combining the saturated solution of the payload with the solution of helper molecules. The combined saturated solution of the payload with the solution of helper molecules is mixed with an aqueous solution to form a water soluble microstructure. The water soluble microstructure includes the helper molecules forming an outside portion of the microstructure, and the payload having poor water solubility contained within the outside portion and forming an inside portion of the microstructure.

### CROSS-REFERENCE TO RELATED APPLICATION

This application claims the benefit of U.S. Provisional Application No. 61/109,013, filed 28 Oct. 2008.

### FIELD OF THE INVENTION

This invention relates to structure and method for preparing water soluble microstructures from water insoluble molecules.

### BACKGROUND OF THE INVENTION

In the field of payload delivery in an organic system, such as drugs, problems of great concern are solubility in water and bioavailability. Many drugs and other payloads are oil soluble and suffer from poor bioavailability and can further suffer from delivery problems. The problem of bioavailability has been overcome to some extent by the use of liposome or polymer encapsulated drugs. These, however, can be difficult to fabricate, can be expensive and may be detrimental to the payload.

It would be highly advantageous, therefore, to remedy the foregoing and other deficiencies inherent in the prior art.

Accordingly, it is an object of the present invention to provide new and improved water soluble microstructures.

It is a further object of the present invention to provide new and improved water soluble microstructures to improve bioavailability of a payload having poor water solubility.

### SUMMARY OF THE INVENTION

Briefly, to achieve the desired objects and advantages of the instant invention, provided is a water soluble microstructure including helper molecules forming an outside portion of a microstructure. The helper molecules each having a hydrophilic head directed outwardly. A hydrophobic payload is contained within the outside portion and forms an inside portion of the microstructure.

In a specific aspect the water soluble microstructure is enclosed by a lipid bilayer.

Also provided is a method of preparing a water soluble microstructure. Preparation includes the steps of providing a saturated solution of a payload with poor water solubility, providing a solution of helper molecules, and combining the saturated solution of the payload with the solution of helper molecules. The combined saturated solution of the payload with the solution of helper molecules is mixed with an aqueous solution to form a water soluble microstructure. The water soluble microstructure includes the helper molecules forming an outside portion of the microstructure, and the payload having poor water solubility contained within the outside portion and forming an inside portion of the microstructure.

In a specific aspect of the method, a step of enclosing the water soluble microstructure in a lipid bilayer is included. The step includes providing water soluble microstructures, providing liposomes, and combining the water soluble microstructures and liposomes at elevated temperatures, above the lipid transition temperatures, and using ultrasound to induce the liposomes to nucleate on the water soluble microstructures and fuse, forming a continuous lipid bilayer.

### BRIEF DESCRIPTION OF THE DRAWINGS

The foregoing and further and more specific objects and advantages of the instant invention will become readily apparent to those skilled in the art from the following detailed description of a preferred embodiment thereof taken in conjunction with the drawings, in which:

FIG. 1 is a schematic depiction of solid antioxidant statin microparticles; and

FIG. 2 illustrates a typical light scattering size distribution bar graph of microparticles for atorvastatin.

### DETAILED DESCRIPTION OF A PREFERRED EMBODIMENT

In this new structure and method for preparing water soluble microstructures from statin drugs which have poor water solubility, a key element is a process by which the statin drugs are dissolved in ethanol or other water miscible solvents such as acetone, isopropanol, methanol and the like, including alcohols, ketones, amides, amines, etc. After forming a saturated solution, the statin drug is combined with a helper material. The combination of solutions is then mixed with an aqueous solution that may comprise water, saline, ionic, or various buffer solutions. The resulting turbid mixture is vigorously mixed, and can be further combined with water to dilute the solution and achieve the smallest particle size and optimum monodispersity. The microstructures that result are solid, with the statin generally in the inner portion of the particle with the helper material arranged into its most hydrophilic orientation. It will be understood that other hydrophobic, small molecule drugs can be formed into microstructures using the same process.

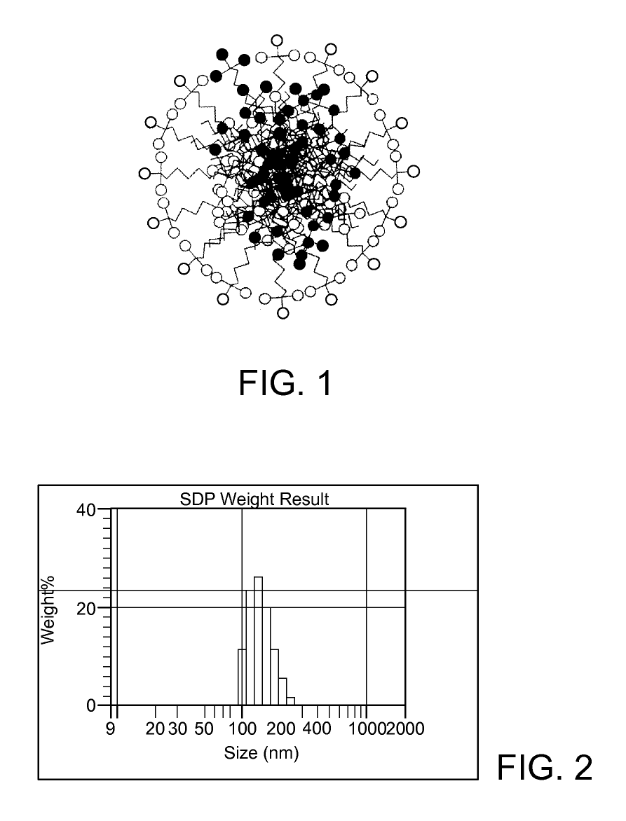

Statins such as atorvastatin (Lipitor), simvastatin (Zocor), fluvastatin, and others have poor water solubility and bioavailability. A new water soluble form of these statins is desirable to enhance bioavailability and improve stability. By combining statins with small amounts of a helper molecule such as the antioxidants tocopherol, coenzyme-Q10, etc., the statins can be caused to self-assemble into a stable, water soluble form comprising microparticles with a size in the range of 100-1000 nm. The microparticles form and become water soluble over a range of percentages of helper molecules from 50% to 10% or lower, by weight. A possible structure is illustrated in FIG. 1, whereby the helper molecule (white) is predominantly situated at the outside of the nanoparticle with its more hydrophilic head group pointed towards the aqueous solution. Similarly, the hydrophobic drug payload (black) is predominantly contained within the interior of the microparticle.

In order for the mixture to form water soluble nanoparticles, the helper molecules must generally have a chain-like hydrocarbon structure with a more polar group (usually OH) at one or both ends so that the molecules tend to align at the surface of the particles in such a manner that their least hydrophobic end is at the surface interacting with the water.

To form the drug microparticles, the drug is first dissolved in a water miscible polar solvent such as ethanol, acetone, methanol, isopropanol and the like. Depending on the drug and its desired concentration, the solvent is heated to elevated temperatures below the melting point of the drug (as high as 50-100 C) to increase the solubility. After the drug has dissolved into the solvent and formed a transparent solution, it is combined with a solvent solution containing the helper material. In the case of atorvastatin, for example, a volume of drug at a concentration of 50 mg/ml is combined with varying concentrations (10-50 mg/ml) and amounts of tocopherol in alcohol to reach final ratios of drug/helper in the range of 1:1 to 20:1. A volume of the combined solutions are then streamed into a volume, typically equal, of water or aqueous buffer and vortexed vigorously. The volume of combined solutions and water or aqueous buffer is then further combined with an additional volume of water and vortexed once again. Since the drug/helper mix is not soluble in the solvent-water mixture, it rapidly forms solid microparticles. These particles range from 100-1000 nm in size and are monodisperse.

For the case of atorvastatin (Lipitor) and tocopherol at a ratio by weight of 10:1, microparticles were formed with a size of 163 nm and a standard deviation of 43 nm, as determined by dynamic light scattering shown in FIG. 2.

Similar results have been obtained with simvastatin (Zocor). The statin microparticles remain stable in the aqueous solution for long periods of time at room temperature, indicating that the microparticles are small and water soluble.

The helper lipids can be chosen from materials that potentially have a synergistic effect with the statins or other hydrophobic drugs. For example, while water solubility is generally good for bioavailability, for cellular uptake, lipophilic characteristics are helpful. Thus, when the microparticles dissolve and release the active drug, proper choice of the antioxidant molecule may enhance cellular uptake. Also, statins and other drugs are known to deplete key enzymes in the body. In particular, statins cause a depletion of coenzyme-Q10 in the cells, which has been proposed as a cause for the muscle aches and memory impairment sometimes associated with statin use. By using CoQ10 as the helper molecule, it is possible to prevent a depletion of CoQ10 in the cells and reduce an undesirable side effect of using a statin drug. Other synergistic drug combinations are readily available, as well, including such materials as vitamin E, vitamin A, lutein, Lycopene, beta carotene, omega fatty acids, resveratrol, and the like. Higher fractions of the helper molecules may be utilized in these cases, even to the extent whereby the number of helper molecules exceeds the number of drug molecules. Particles with more than two components can be formulated as well using a straightforward extension of the process. Microparticles with fractions of helper molecules as high as 50% have been demonstrated.

HPLC and differential scanning calorimetry measurements on purified samples show that the statins have been successfully incorporated into the microparticles.

These new microparticles are also widely applicable to other hydrophobic drugs that can be dissolved in solvents that are miscible with water.

Another composition of matter comprises the drug containing water soluble microparticle, enclosed with a lipid bilayer. This forms a stable microparticle with the desirable surface membrane properties of liposomes, but in a more stable solid geometry. The lipid bilayers serve to enhance stability in the bloodstream and are readily synthesized with targeting moieties for targeted therapies.

In addition, liposomes tend to accumulate at the liver, which is the target organ for statins, thereby leading to enhanced bioavailability. The lipid membranes can also include PEG or other stealth components to increase circulation time in the bloodstream and minimize the first pass metabolism effects. The two step process to produce lipid encapsulated supplements utilizes water soluble microparticles and liposomes that are formed in separate implementations of the ethanol extraction process. The particles are then combined, and at elevated temperatures (above the lipid transition temperatures, typically 50-60 C) and using ultrasound, the liposomes can be induced to nucleate on the nanoparticles and fuse, forming a continuous membrane. This process could have an advantage for delivery of the active drug, whereby targeting moieties and stealth components could be incorporated within the lipid membrane. The structures would thus demonstrate the desirable properties of liposomes for in vivo drug delivery, while providing more stable particles for transport within the body.

As an alternative to lipids, co-block polymers with amphiphilic features, typically used to form polymerosomes, can be used. As a yet another alternative to lipids, bovine serum albumin, biotin, or other moieties may be used as a means to attach specific targeting molecules.

It may desirable for some chemical and analytical applications to make water soluble chemicals more soluble in solvents. A similar approach could be used. Water soluble helper molecules with hydrocarbon tails could be combined with water soluble payload (for example, vitamin C) in water, and then injected into solvent in such a manner that the hydrocarbon tails on the helper molecules would point outward, rendering the microparticle oil (solvent) soluble.

Various changes and modifications to the embodiments herein chosen for purposes of illustration will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. A water soluble microstructure comprising:
helper molecules forming a substantially outside portion of a microstructure, the helper molecules each having a hydrophilic head directed outwardly; and
a hydrophobic payload contained predominantly within the outside portion and forming an inside portion of the microstructure.

2. A water soluble microstructure as claimed in claim 1 wherein the helper molecules include an antioxidant.

3. A water soluble microstructure as claimed in claim wherein the helper molecules is selected from a group including tocopherol, coenzyme-Q10, vitamin E, vitamin A, lutein, lycopene, beta carotene, and omega fatty acids.

4. A water soluble microstructure as claimed in claim wherein the helper molecules include a chain-like hydrocarbon structure with a polar group at one end forming the hydrophilic head.

5. A water soluble microstructure as claimed in claim 1 wherein the hydrophobic payload includes drugs with poor water solubility.

6. A water soluble microstructure as claimed in claim wherein the drugs with poor water solubility include statins.

7. A water soluble microstructure as claimed in claim further including a lipid bilayer enclosing the outside portion and the inside portion.

8. A water soluble microstructure as claimed in claim 1 wherein the microstructure has a size in the range of 100-1000 nm.

9. A method of preparing a water soluble microstructure comprising the steps of:
providing a saturated solution of a payload with poor water solubility;
providing a solution of helper molecules;
combining the saturated solution of the payload with the solution of helper molecules; and
mixing the combined saturated solution of the payload with the solution of helper molecules in an aqueous solution to form a water soluble microstructure including the helper molecules forming an outside portion of the microstructure, and the payload having poor water solubility contained substantially within the outside portion and forming an inside portion of the microstructure.

10. A method as claimed in claim 9 wherein the step of providing a saturated solution of the payload with poor water solubility includes:
providing a water miscible solvent; and
dissolving the payload with poor water solubility in the water miscible solvent.

11. A method as claimed in claim 10 wherein the step of providing the water miscible solvent further includes selecting from a group consisting of alcohols, ketones, amides, and amines.

12. A method as claimed in claim 10 wherein the step of dissolving further includes heating the water miscible solvent to elevated temperatures below the melting point of the payload.

13. A water soluble microstructure as claimed in claim 10 wherein the payload with poor water solubility includes statins.

14. A method as claimed in claim 9 wherein the step of providing a solution of helper molecules includes:
providing a water miscible solvent; and
dissolving the helper molecules in the water miscible solvent.

15. A method as claimed in claim 14 wherein the step of providing the helper molecules includes providing an antioxidant.

16. A method as claimed in claim 14 wherein the step of providing the helper molecules includes selection from a group including tocopherol, coenzyme Q10, vitamin E, vitamin A, lutein, lycopene, beta carotene, and omega fatty acids.

17. A method as claimed in claim 14 wherein the step of providing the helper molecules includes providing a chain-like hydrocarbon structure with a polar group at one end forming a hydrophilic head.

18. A method as claimed in claim 9 wherein the step of mixing includes forming water soluble microstructures having a size in the range of 100-1000 nm.

19. A method as claimed in claim 9, further comprising the step of enclosing the outside portion and the inside portion with a lipid bilayer.

20. A method as claimed in claim 19 further wherein the step of enclosing further includes the steps of:
providing water soluble microstructures;
providing liposomes;
combining the water soluble microstructures and liposomes at elevated temperatures, above the lipid transition temperatures, and using ultrasound to induce the liposomes to nucleate on the water soluble microstructures and fuse, forming a continuous lipid bilayer.
